(19) United States
(12) Patent Application Publication
(10) Pub. No.: US 2004/0005661 A1
(43) Pub. Date: Jan. 8, 2004

(54) Potential growth factors from the human tumour cell line ht 1080

(76) Inventors: Stephen L. Minger, London (GB); Gregor Adams, London (GB); Paul Francis, London (GB); Myra Mcclure, London (GB)

Correspondence Address: MARY M. KRINSKY, Ph. D., J.D., PATENT ATTORNEY, 79 TRUMBULL STREET, NEW HAVEN, CT, 06511, US

(21) Appl. No.: 10/344,503

(22) PCT Filed: Aug. 6, 2001
(86) PCT No.: PCT/GB01/03523

(30) Foreign Application Priority Data
Aug. 10, 2000 (GB) 0019705.3

(51) Int. Cl. C12N 9/64; C07H 21/04; C12N 5/08; C07K 14/47; C12P 21/02

(52) U.S. Cl. 435/69.1; 435/226; 435/320.1; 435/366; 530/350; 536/23.2

(57) ABSTRACT

The invention relates to a mitogen obtainable from a human tumour cell line, such as from HT1080 cells.

### FIELD OF THE INVENTION

The present invention relates to mitogenic factor(s). In particular, the present invention relates to factor(s) having or potentiating a mitogenic effect on stem cells, such as neural stem cells (NSCs).

### BACKGROUND TO THE INVENTION

Culturing cells in vitro requires a numerous factors to be supplied in the medium. These factors include buffers, glucose, proteins, serum, salts etc. These factors also include signalling molecules such as growth factors, survival factors and related molecules necessary for the successful propagation of the particular cell line.

The number of different growth factors/survival factors which are available commercially is small.

Numerous cell lines are difficult to propagate in culture, often because appropriate factors for their culture are not available.

Many comercially available growth factors are expensive to purchase and/or labour-intensive to produce.

Neural cells or stem cells are difficult to culture in vitro. Known mitogens are often ineffective in promoting the propagation of such cells. For example, EGF, leukemia inhibitory factor (LIF, 10-20 ng/ml), granulocyte-macrophage colony stimulatory factor (GM-CSF, 1-10 ng/ml), and vascular endothelial growth factor (VEGF; 10 ng/ml) are poor mitogens for forebrain neural stem cells. In particular, expansion of neurotransmitter-specific neuronal populations is a problem of neural stem cell research.

### SUMMARY OF THE INVENTION

It is surprisingly shown herein that conditioned medium from a human tumour cell line is capable of acting as a potent mitogen. This mitogen is termed NSC-MF.

In addition to its mitogenic properties, NSC-MF potentiates the effects of other stem cell mitogens, such as neural stem cell (NSC) mitogens, including fibroblast growth factor-2 (FGF-2) and leukemia inhibitory factor (LIF).

Thus, the invention relates to NSC-MF and to the use of NSC-MF as a mitogen.

In another aspect, the invention relates to the use of NSC-MF as a potentiator of other mitogen(s).

As used herein, the term ‘NSC-MF’ refers to the mitogen itself and/or to conditioned medium or cell supernatent comprising the NSC-MF mitogen.

### SUMMARY ASPECTS OF THE PRESENT INVENTION

The present invention is based on the finding that NSC-MF has mitogenic properties.

The methods of the present invention utilise this finding. It enables cells to be propagated in vitro in reduced levels of, in subset(s) of, or even in the absence of, conventional mitogens or growth factors.

### DETAILED ASPECTS OF THE PRESENT INVENTION

In one aspect, the present invention relates to an isolated or purified mitogen obtainable from a human tumour cell line.

In another aspect, the present invention relates to conditioned medium comprising a mitogen, said medium being conditioned via the growth of a human tumour cell line therein.

In another aspect, the present invention relates to conditioned medium comprising a mitogen, said medium being conditioned via the growth of a human tumour cell line, such as HT1080 cells (ATCC number ATCC #CCI-121) therein.

In another aspect, the present invention relates to an isolated or purified mitogen obtainable from human tumour cell line, such as HT1080 cells (ATCC number ATCC #CCI-121).

In another aspect, the present invention relates to a mitogen obtainable from the medium in which a human tumour cell line, such as HT1080 cells (ATCC number ATCC #CCI-121) has been cultured.

In another aspect, the present invention relates to a stem cell mitogen obtainable from human tumour cell line, such as HT1080 cells (ATCC number ATCC #CCI-121).

In another aspect, the present invention relates to a stem cell mitogen obtainable from the medium in which a human tumour cell line, such as HT1080 cells (ATCC number ATCC #CCI-121) has been cultured.

In another aspect, the present invention relates to a neural stem cell mitogen obtainable from human tumour cell line, such as HT1080 cells (ATCC number ATCC #CCI-121).

In another aspect, the present invention relates to a neural stem cell mitogen obtainable from the medium in which a human tumour cell line, such as HT1080 cells (ATCC number ATCC #CCI-121) has been cultured.

In another aspect, the present invention relates to a method for culturing eukaryotic cells in vitro in a medium comprising a mitogen as described herein.

In another aspect, the present invention relates to a method for culturing eukaryotic cells in vitro in a medium comprising an exogenously added mitogen as described herein.

In another aspect, the present invention relates to a method for culturing eukaryotic cells in vitro in a medium comprising exogenously added conditioned medium as described herein.

In another aspect, the present invention relates to a method for promoting cell survival comprising culturing said cell in medium comprising an exogenously added mitogen as described herein.

In another aspect, the present invention relates to a method for promoting cell survival comprising culturing said cell in medium comprising exogenously added conditioned medium as described herein.

In another aspect, the present invention relates to a mitogen as described herein wherein said mitogen is a protein or fragment thereof.

In another aspect, the present invention relates to a mitogenic composition comprising a mitogen as described herein, and/or comprising conditioned medium as described herein.

In another aspect, the present invention relates to a method for the preparation of a mitogenic composition comprising

(i) providing a suitable diluent

(ii) adding a mitogen and/or conditioned medium as described herein thereto.

In another aspect, the present invention relates to a method for modulating the mitogenic effect of a first mitogen comprising adding a suitable amount of a second mitogen wherein said second mitogen is a mitogen according to the present invention as described herein.

In another aspect, the present invention relates to a method for augmenting the mitogenic effect of a factor selected from GM-CSF, LIF, EGF, IGF-1, FGF, Thrombopoietin, neurotrophin-3, or FGF-8, comprising adding an augmenting amount of a mitogen as described herein.

In another aspect, the present invention relates to a method for propagating a cell, said method comprising incubating said cell in a medium comprising a mitogen as described herein.

In another aspect, the present invention relates to a method for potentiating neural cell growth as shown in the accompanying drawings comprising the use of a mitogen according to the invention as described herein. Preferably such cells have morphologies as exemplified in the figures, such as FIGS. 4B and/or 5B. Preferably, said potentiation is of similar magnitude to that shown in FIG. 1 and/or FIG. 2 and/or FIG. 3.

In another aspect, the present invention relates to the use of a mitogen according to any previous claim to selectively potentiate the proliferation of progenitor cells that give rise to an increase in neurons.

An example of such an effect may be found in the culturing of freshly dissected forebrain NSCs in medium comprising NSC-MF as compared to the culturing of said cells in medium not comprising NSC-MF. The cells cultured in medium comprising NSC-MF increase in number as discussed herein, and preferably also those cells which give rise to neurons are selectively potentiated. This effect may be assessed by comparing the proportion of cells developing into neurons when cultured in medium comprising NSC-MF mitogen according to the present invention with the proportion of cells developing into neurons when said mitogen is not comprised in the culture medium. By comparing the proportions in this manner, the advantageous effects of NSC-MF in increasing the proliferation of said cells do not bias the comparison of the advantageous feature of NSC-MF increasing the proportion of cells giving rise to neurons as discussed herein. For example, propagating freshly dissected forebrain NSCs in medium comprising EGF can result in 2-5% neuronal progenitor cells, propagating freshly dissected forebrain NSCs in medium comprising FGF can result in 20% neuronal progenitor cells, whereas propagating freshly dissected forebrain NSCs in medium comprising NSC-MF can result in more than 20%, preferably more than 30% neuronal progenitor cells, or even more. Further details may be found below, such as in the Examples section.

Thus, in another aspect, the present invention relates to a method of increasing the proportion of neurons developing from a population of neural stem cells, said method comprising contacting said population of neural stem cells with a mitogen according to any previous claim.

For ease of reference, these and further aspects of the present invention are now discussed under appropriate section headings. However, the teachings under each section are not necessarily limited to each particular section.

Preferable Aspects

Preferably, the invention relates to use of NSC-MF as a mitogen for eukaryotic cell population(s).

In a preferred embodiment, the invention relates to the use of NSC-MF as a mitogen for stem cell population(s).

In a highly preferred embodiment, the invention relates to use of NSC-MF as a mitogen for NSCs.

Advantages

It is an advantage of the present invention that, by using NSC-MF in the culture of cells in vitro, reduced quantities of conventional mitogens may be used.

It is an advantageous feature of aspects of the present invention that, by using NSCMF in the culture of certain cells in vitro, fewer other mitogens need be exogenously added.

It is an advantageous feature of aspects of the present invention that, by using NSCMF in the culture of certain cells in vitro, cell survival rates are enhanced.

It is an advantageous feature of aspects of the present invention that, by using NSCMF in the culture of certain cells in vitro, cell mortality rates are decreased.

It is an advantageous feature of aspects of the present invention that, by using NSCMF in the culture of certain cells in vitro, said cells may be cultured at lower initial densities.

It is an advantageous feature of aspects of the present invention that, by using NSCMF in the culture of certain cells in vitro, higher rates of cellular proliferation (eg. higher expansion rates) may be produced.

It is an advantageous feature of aspects of the present invention that, by using NSCMF in the culture of certain cells in vitro, a more neuronal population of cells may be produced.

It is an advantageous feature of aspects of the present invention that, by using NSCMF in the culture of certain cells in vitro, maintenance of primary cells in culture is facilitated.

NSC-MF Polypeptide(s)

NSC-MF can be supernatent or an active fraction thereof or an active component of any thereof. NSC-MF can also include mixtures of NSC-MFs. As used herein, ‘active’ means capable of acting as a mitogen as described herein.

Disclosed herein is the isolation of NSC-MF. Furthermore, its purification is described.

NSC-MF may be isolated and/or optionally concentrated and/or optionally purified from conditioned medium using protein chemical techniques. Such techniques are well known in the art, in particular use may be made of techniques used in protein chemistry. For example, the serum-free conditioned medium may be freeze dried, and desalted by dialysis. The extract may then be fractionated by liquid chromatography (LC) and the extracts analysed for mitogenic activity using proliferation assays of forebrain NSCs; these techniques are discussed in more detail below, such as in the Examples section.

This process may be repeated until a relatively clean fraction of the active constituent(s) is obtained. At this stage it is desirable to carry out 2D electrophoresis on the fractions. The peptides may be chemically or enzymatically cleaved and the peptide mixture from the cleavage on the gel may be subjected directly to matrix assisted laser desorption mass spectrometry (MALDI-MS).

Such data can be evaluated on-line so that the protein can be identified with a high probability in sequence data banks from the peptide mass pattern obtained (f the protein is known). Identification can be ambiguous with some proteins and further analysis can be carried out in order to clarify their identity (e.g. Electrospray ionisation MS).

Using the sequence of the peptide, it may be chemically synthesised as is well known in the art. Alternatively, or in addition, the peptide may be produced using recombinant DNA technology.

Using peptide sequence information from the MS analysis, the NSC-MF cDNA may be isolated and sequenced.

In this manner, NSC-MF may be isolated, it's amino-acid sequence determined, and the protein(s) characterised according to any suitable methods known in the art. This is discussed in more detail below.

It is believed that the mitogen of the present invention has an approximate molecular weight selected from 8.7, 10.1, 11.8, 12.5, 13.6, 33.5 45.3 and 67.1 kilodaltons (kDa).

In one preferred aspect, the mitogen of the present invention has a molecular weight of either greater than 30 kD (which we sometimes refer to as “NSC-MF-1”) or less than 10 kD (which we sometimes refer to as “NSC-MF-2”).

In a preferred aspect, the mitogen of the present invention is obtainable from HT1080 cells (ATCC #CCI-121).

Preferably the mitogen is obtainable from a method comprising the use of sequential Sepharose 4B and anion exchange chromatographic techniques to at least partially purify the mitogen from other contaminating proteins.

In a preferred aspect, the mitogen of the present invention is obtainable from HT1080 cells (ATCC #CCI-121) which have been grown in DMEM medium (Gibco) with 10% FCS (without antibiotics).

In a preferred aspect, the mitogen of the present invention is obtainable from a method comprising growing HT1080 cells (ATCC #CCI-121) in DMEM medium (Gibco) with 10% FCS (without antibiotics); and wherein when said cells are approximately 90% confluent, removing said serum-containing medium and exposing said cells to serum-free medium.

In a preferred aspect, the mitogen of the present invention is obtainable from a method comprising growing HT180 cells (ATCC #CCI-121) in DMEM medium (Gibco) with 10% FCS (without antibiotics); and wherein when said cells are approximately 90% confluent, removing said serum-containing medium and exposing said cells to serum-free medium for 3648 hours.

In a preferred aspect, the mitogen of the present invention is obtainable from a method comprising growing HT1080 cells (ATCC #CCI-121) in DMEM medium (Gibco) with 10% FCS (without antibiotics); and wherein when said cells are approximately 90% confluent, removing said serum-containing medium and exposing said cells to serum-free medium (preferably for 3648 hours); removing said medium; centrifuging; and collecting the supernatant collected from the pelleted cells.

In a preferred aspect, the mitogen of the present invention is obtainable from a method comprising growing HT1080 cells (ATCC #CCI-121) in DMEM medium (Gibco) with 10% FCS (without antibiotics); and wherein when said cells are approximately 90% confluent, removing said serum-containing medium and exposing said cells to serum-free medium (preferably for 3648 hours); removing said medium; centrifuging; collecting the supernatant collected from the pelleted cells; and filtering said medium.

Preferably said mitogen is secreted into the tissue culture medium, is sensitive to heating at 100° C. for five minutes, and is retained following filtration through a 0.22 μm filter.

Expression

The mitogen of the invention (NSC-MF) may be produced using recombinant DNA technology, for example by placing the cDNA encoding it into a suitable expression vector. Expression and cloning vectors usually contain a promoter that is recognised by the host organism and is operably linked to mitogen (NSC-MF) encoding nucleic acid. Such a promoter may be inducible or constitutive. The promoters are operably linked to DNA encoding the mitogen (NSC-MF) by removing the promoter from the source DNA by restriction enzyme digestion and inserting the isolated promoter sequence into the vector. Both the native mitogen (NSC-MF) promoter sequence and many heterologous promoters may be used to direct amplification and/or expression of mitogen (NSC-MF) encoding DNA.

Promoters suitable for use with prokaryotic hosts include, for example, the β-lactamase and lactose promoter systems, alkaline phosphatase, the tryptophan (Trp) promoter system and hybrid promoters such as the tac promoter. Their nucleotide sequences have been published, thereby enabling the skilled worker operably to ligate them to DNA encoding nucleic acid binding protein, using linkers or adapters to supply any required restriction sites. Promoters for use in bacterial systems will also generally contain a Shine-Delgarno sequence operably linked to the DNA encoding the nucleic acid binding protein.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21 (DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcHisXpress™ (Invitrogen) or pTrc99 (Pharmacia Biotech, SE) or vectors containing the tac promoter such as pKK223-3 (Pharmacia Biotech) or PMAL (New England Biolabs, Mass., USA).

Moreover, the mitogen (NSC-MF) gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate. A “leader” peptide may be added to the N-terminal finger. Preferably, the leader peptide is MAEEKP.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3 phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (-173) promoter element starting at nucleotide-173 and ending at nucleotide-9 of the PH05 gene.

Mitogen (NSC-MF) gene transcription from vectors in mammalian hosts may be controlled by promoters derived from the genomes of viruses such as polyoma virus, adenovirus, fowipox virus, bovine papilloma virus, avian sarcoma virus, cytomegalovirus (CMV), a retrovirus and Simian Virus 40 (SV40), from heterologous 5 mammalian promoters such as the actin promoter or a very strong promoter, e.g. a ribosomal protein promoter, and from the promoter normally associated with mitogen (NSC-MF) sequence, provided such promoters are compatible with the host cell systems.

Transcription of a DNA encoding mitogen (NSC-MF) by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to mitogen (NSCMF) DNA, but is preferably located at a site 5′ from the promoter.

Advantageously, a eukaryotic expression vector encoding a mitogen (NSC-MF) according to the invention may comprise a locus control region (LCR). LCRs are capable of directing high-level integration site independent expression of transgenes integrated into host cell chromatin, which is of importance especially where the mitogen (NSC-MF) gene is to be expressed in the context of a permanently-transfected eukaryotic cell line in which chromosomal integration of the vector has occurred, or in transgenic animals.

Eukaryotic vectors may also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding nucleic acid binding protein.

An expression vector includes any vector capable of expressing mitogen (NSC-MF) nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding mitogen (NSC-MF) may be inserted into a vector suitable for expression of cDNAs in mammalian cells, e.g. a CMV enhancer-based vector such as pEVRF (Matthias, et al., (1989) NAR 17, 6418).

Particularly useful for practising the present invention are expression vectors that provide for the transient expression of DNA encoding mitogen (NSC-MF) in mammalian cells. Transient expression usually involves the use of an expression vector that is able to replicate efficiently in a host cell, such that the host cell accumulates many copies of the expression vector, and, in turn, synthesises high levels of nucleic acid binding protein. For the purposes of the present invention, transient expression systems are useful e.g. for identifying mitogen (NSC-MF) mutants, to identify potential phosphorylation sites, or to characterise functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing mitogen (NSC-MF) expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

In accordance with another embodiment of the present invention, there are provided cells containing the above-described nucleic acids. Such host cells such as prokaryote, yeast and higher eukaryote cells may be used for replicating DNA and producing the nucleic acid binding protein. Suitable prokaryotes include eubacteria, such as Gram-negative or Gram-positive organisms, such as E. coli, e.g. E. coli K-12 strains, DH5a and HB101, or Bacilli. Further hosts suitable for the mitogen (NSC-MF) encoding vectors include eukaryotic microbes such as filamentous fungi or yeast, e.g. Saccharomyces cerevisiae. Higher eukaryotic cells include insect and vertebrate cells, particularly mammalian cells including human cells or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of the nucleic acid binding protein-encoding nucleic acid to form the nucleic acid binding protein. The precise amounts of DNA encoding the mitogen (NSC-MF) may be empirically determined and optimised for a particular cell and assay.

Host cells are transfected or, preferably, transformed with the above-captioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby the mitogen (NSC-MF) encoded by the DNA is expressed. The composition of suitable media is known to those in the art, so that they can be readily prepared. Suitable culturing media are also commercially available.

In another aspect, the administration of a nucleic acid construct capable of directing the expression of NSC-MF will be accomplished using a vector, preferably a viral vector, more preferably a retroviral vector. In a highly preferred embodiment, the administration of a nucleic acid construct capable of directing the expression of NSC-MF will be accomplished using a retroviral vector capable of infecting non-dividing mammalian cells such as neural cells.

Polynucleotides of the invention may introduced into suitable host cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Molecules of the invention may introduced into suitable host cells using a delivery system. The delivery system may be a viral delivery system. Viral delivery systems include but are not limited to adenovirus vector, an adeno-associated viral (MV) vector, a herpes viral vector, retroviral vector, lentiviral vector, baculoviral vector.

Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, gene delivery is typically mediated by viral infection of a target cell.

Retroviral Vectors

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

Preferred vectors for use in accordance with the present invention are recombinant viral vectors, in particular recombinant retroviral vectors (RRV) such as lentiviral vectors.

The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV carries non-viral coding sequences which are to be delivered by the vector to the target cell. An RRV is incapable of independent replication to produce infectious retroviral particles within the final target cell. Usually the RRV lacks a functional gag-pol and/or env gene and/or other genes essential for replication.

A detailed list of retroviruses may be found in Coffin et al (“Retroviruses” 1997 Cold Spring Harbour Laboratory Press Eds: J M Coffin, S M Hughes, H E Varmus pp 758-763).

Alternatively, the delivery system may be a non-viral delivery system—such as by way of example DNA transfection methods of, for example, plasmids, chromosomes or artificial chromosomes. Here transfection includes a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Assays

Any one or more of appropriate targets—such as an amino acid sequence and/or nucleotide sequence—may be used for identifying an agent capable of modulating or interacting with NSC-MF in any of a variety of drug screening techniques. The target employed in such a test may be free in solution, affixed to a solid support, borne on a cell surface, or located intracellularly. The abolition of target activity or the formation of binding complexes between the target and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WOA-84/03564.

It is expected that the assay methods of the present invention will be suitable for both small and large-scale screening of test compounds as well as in quantitative assays.

In one preferred aspect, the present invention relates to a method of identifying agents that selectively modulate or interact with NSC-MF.

Another example of an assay that may be used is described in WOA-9849271, which concerns an immortalised human terato-carcinoma CNS neuronal cell line, which is said to have a high level of neuronal differentiation and is useful in detecting compounds which bind to NSC-MF.

Mitogenic Properties of NSC-MF

NSC-MF's potential to act as a mitogen for Neural Stem Cell (NSC) populations may be determined as set out herein. Without wishing to be bound by theory, we believe that NSC-MF may have mitogenic effects on other Stem Cell populations, as well as other cell populations. For example, it is envisaged that NSC-MF may have mitogenic effect(s) on muscle cells or muscle cell derived cell line(s), and may also have mitogenic effec(s) on other cell type(s).

Occasionally NSC-MF may be referred to herein, such as in the Figures, as Proliferin, Factor X or F-X. These terms are synonymous and refer to NSC-MF according to the present invention.

It is desirable to assess the full mitogenic potential of NSC-MF by examining it's effect on other stem cell populations.

As disclosed herein, NSC-MF is useful as a mitogen. It is further disclosed herein that NSC-MF is useful in the potentiation of other mitogenic signals. Potentiation may include the use of lower concentrations of a particular factor in the prescence of NSC-MF than would be required in the absence of NSC-MF, or may include a synergistic effect, ie. where the use of NSC-MF in addition to another factor produces an effect greater than the sum of the effect of the two factors used separately. Potentiation may include the modulation, enhancement, increase or alteration of one or more of the effect(s) of a mitogenic signal or mitogen. The terms potentiation and augmentation are used synonymously herein.

NSC-MF is a neural stem cell mitogenic factor.

The identity of NSC-MF is understood in terms of its characteristics which are disclosed herein.

The amino acid sequence of NSC-MF is obtainable as described herein.

In a preferred embodiment, NSC-MF is synergistic with one or more other stem cell mitogen(s) such as LIF or GM-CSF. In a highly preferred embodiment, NSC-MF significantly increases the proliferative potential of other stem cell populations such as human embryonic stem cells, hematopoietic stem cells, and other CNS neural stem cell populations.

According to the invention, it may be determined whether stem cell populations are responsive to NSC-MF. By way of example, this is accomplished via the detection of mitogenic activity in the same fashion as the effects of NSC-MF on forebrain NSCs are demonstrated, eg. using conditioned medium (ie. comprising NSC-MF) from the human tumour cell line HT 1080.

NSC-MF promotes the proliferation of NSCs from the rat ventral forebrain.

Importantly, NSC-MF appears to increase the number of progenitor cells that give rise to neurons compared to other mitogens. Thus, NSC-MF preferentially stimulates neuronal progenitor cells.

The effects of NSC-MF, both alone and in combination with other NSC mitogens, may be assessed on one or more of the following NSC populations, or on any other suitable cell population:

ventral mesencephalon—This region of the brain is a source of dopamine (DA) containing neurons, the same neurons that are selectively lost in Parkinson's disease. Given that transplantation of foetal dopamine-containing cells significantly improves the symptoms of Parkinson's patients, there is a major effort, both commercial and academic, to find conditions that would enrich for DA neurons in vitro.

To date, there has been an almost complete failure to derive dopamine-containing neurons from either rodent or human neural stem cell cultures; one of the important considerations seems to be the result of inappropriate culture conditions (e.g. lack of a crucial factor). NSC-MF may promote the proliferation of DA neuronal progenitor cells.

spinal cord—Limited proliferation of spinal cord NSCs is observed with FGF-2, whilst more substantial proliferation is observed when FGF-2 is combined with crude chick embryo extract (CEE). This potentiation of FGF-2 by CEE may be similar to that observed with FGF-2 and NSC-MF with forebrain NSCs. Therefore, NSC-MF may have similar effects on spinal cord stem cells. Given the interest in functional restoration of spinal cord damage and the potential for spinal cord transplantation, it is clearly desirable to determine the extent to which NSC-MF promotes spinal cord NSC proliferation.

Tissue culture supplies (e.g. plastics, laminin, N2 supplement, FGF-2) are as commonly commercially available in the art. Both mesencephalic and spinal cord neural stem cells can be obtained from foetal animals as used in forebrain NSC cultures. Methods for the propagation of these cells (ie. culture conditions etc.) are as used for forebrain stem cell cultures, with modifications as discussed herein where necessary.

NSC-MF potentiates the mitogenic effects of LIF on NSCs. Importantly, LIF is also an obligatory mitogenic factor for mouse Embryonic Stem (ES) cells, keeping ES cells in an undifferentiated state. ES cells represent the most primitive totipotent stem cells, and in principle could give rise to all tissue-specific stem cell populations, and therefore to all cell types throughout the organism. Totipotent, proliferative human ES cell lines have recently been generated. NSC-MF may be able to substitute for, or potentiate, the mitogenic effects of LIF in ES cell cultures. NSC-MF may be an effective mitogen for mouse ES cells. These properties may be further characterised using techniques discussed herein, and as known in the art.

ES cells may be cultured according to defined protocols (e.g. see Niwa H, Burdon T, Chambers I, Smith A. Self-renewal of pluripotent embryonic stem cells is mediated via activation of STAT3. Genes and Development Jul. 1, 1998; 12(13):2048-60). As these conditions are distinct from neural stem cell cultures, medium, growth factors, and supplements may be purchased separately from the appropriate commercial suppliers. Mouse ES cell lines may be obtained from the ATCC, Specialty Media Company; human ES cell lines are developed by University of Wisconsin (Nature Medicine, 2000, 3, pg 237).

The methods of the present invention may be usefully applied to the creation of modified neuronal cells. Prior art attempts to generate genetically modified neuronal cells, such as transfected neuronal cells, have been relatively unsuccessful. The methods of the present invention may be applied to this problem by culturing the cells in the prescence of a mitogen according to the invention (eg. NNSC-MF) and performing the genetic alteration (eg. transfection such as lipofectamine, electrotransfection, or other suitable technique known to those skilled in the art). This may be usefully applied for example to the production of presenilin mutant neuronal cell line(s). Furthermore, NSC-MF may potentiate retroviral modification of neuronal cells-this is discussed in the appropriate section(s) herein.

The NSC-MF gene may be cloned, sequenced and characterised as discussed herein, and by any other suitable method(s) known in the art.

NSC-MF may be purified to homogeneity and it's amino acid sequence determined. This information facilitates the isolation and sequence determination of the corresponding NSC-MF cDNA and/or gene.

Amino acid sequence information may be used to search protein/DNA databases for sequence homology matches.

Molecular probes may be designed based on the amino acid sequence information. These may be used for example to screen a cDNA library made from the tumour cell line itself or to screen other libraries of interest.

NSC-MF is a novel protein/gene that has mitogenic effects on stem cell populations.

NSC-MF is a potent mitogenic protein for neural stem cells.

Without wishing to be bound by theory, it is believed that the invention may be effective in the following embodiments.

It has recently been shown that ES cells derived from human blastocysts require a feeder layer of mouse fibroblasts for proliferation; the simple addition of LIF to the culture medium is ineffective in the absence of the feeder layer. As the source of NSC-MF is a human fibroblast cell line, it may be that NSC-MF is a requisite factor for human ES cells. In addition, or in the alternative, there may be another factor in the crude conditioned medium that potentiates effect(s) of LIF. The HPLC fractions of medium comprising NSC-MF obtained as described herein and/or NSC-MF alone may cause proliferation of human ES cells.

NSC-MF has mitogenic effects on types of stem cells other than neural stem cells. NSC-MF may have a mitogenic effect on hematopoietic stem cells (HSCs). These cells are capable of multilineage reconstitution of the entire hematopoietic system. HSCs generally proliferate poorly in vitro and complex culture conditions (e.g. growth on stromal cell lines) have been required for HSC proliferation. Recently, several mitogens (e.g. IL-11 and Steel Factor, thrombopoietin) have been shown to support the short-term proliferation of these cells in suspension, but long-term propagation of these cells remains problematic. NSC-MF may act as an HSC-mitogen.

Derivation of tissue-speciffic stem cells populations from parental ES cells may be facilitated using NSC-MF. It has been recently reported that neuronal-like cells may be isolated from ES cell populations, including human ES cells. ES cell-derived NSCs offer numerous advantages over tissue-derived NSCs. In the case of human cells, they obviate the need for obtaining foetal brain tissue such as from early elective abortions. They also have the potential to generate a more multipotent NSC, i.e. one that has the potential to generate more types of CNS cell than NSCs isolated from foetal brain. NSC-MF, either alone or in combination with other known neural stem cell mitogens, may be capable of generating long-term, proliferative NSCs from ES cells. These studies may be performed with mouse ES cells, and/or human cells.

It is known that stem cells may be used to deliver nucleic acid construct(s) in gene therapy applications. In particular, neural stem cells/CNS stem cells may be used in such approaches. An example of this is in the use of CNS stem cells to deliver a gene for IL-4 (Interleukin-4) into mammalian glioblastomas. This led to a reduction in the size of tumours, as well as a decrease in associated mortality (see Nature Medicine, April 2000, p.447). Clearly, the mitogen (NSC-MF) according to the present invention will be of use in the preparation and/or propagation of such stem cells. Said cells may even advantageously comprise the mitogen (NSC-MF) of the present invention.

Furthermore, it is envisaged that the present invention will be useful in identification of receptor(s) for NSC-MF, investigation of the intracellular signalling pathway(s) used by responsive cells, target effector(s) for NSC-MF, NSC-MF induced proliferation in NSCs, as well as related areas.

Study of the receptor and/or signal transduction pathway(s) may offer additional targets for commercial exploitation for example in the areas of cancer therapy, cancer screening, anti-tumour pharmacology, CNS drugs or other related fields.

NSC-MF is a highly important factor and may have myriad cellular effects, depending on the cellular context.

NSC-MF has considerable commercial potential.

The present invention may now be described, by way of example only, in which reference may be made to the following figures:

FIG. 1, which shows a bar chart;

FIG. 2, which shows a bar chart;

FIG. 3, which shows a graph;

FIG. 4, which shows photographs;

FIG. 5, which shows photographs;

FIG. 6, which shows a scatterplot;

FIG. 7, which shows a graph;

FIG. 8, which shows a bar chart;

FIG. 9, which shows photographs;

FIG. 10, which shows a bar chart;

FIG. 11, which shows a bar chart;

FIG. 12, which shows a bar chart;

FIG. 13, which shows a bar chart;

FIG. 14, which shows a bar chart;

FIG. 15, which shows a bar chart;

FIG. 16, which shows a bar chart; and

FIG. 17 shows a MALDI profile.

### BRIEF DESCRIPTION OF THE FIGURES

FIG. 1 shows a bar chart of MTT for FGF-2 and NSC-MF treated cells.

FIG. 2 shows a bar chart of mean proliferation for cells treated with different growth factors.

FIG. 3 shows a graph of cell number vs days in culture for cells cultured with FGF or with NSC-MF.

FIG. 4 shows photographs of MAP-2 immunoreactivity of cells propagated with FGF-2, or with FGF-2 and NSC-MF.

FIG. 5 shows photographs of β-tubulin immunoreactivity of cells propagated with FGF-2, or with FGF-2 and NSC-MF.

FIG. 6 shows a scatterplot of ChAT activity vs days in culture for different cells.

FIG. 7 shows a graph of absorbance at 230 nm vs time for an HPLC chromatographic analysis of NSC-MF.

FIG. 8 shows a bar chart of mean proliferation of cells propagated with FGF-2, FGF+NSC-MF (conditioned medium), FGF+NSC-MF (1000 kDa purification) and FGF+NSC-MF (3500 kDa purification).

FIG. 9 shows photographs of MAP-2 (panels c+d) and β-tubulin (panels a+b) immunoreactivity in cells propagated in FGF alone (panels a+c), or in NSC-MF (conditioned medium) (panels b+d).

FIG. 10. Mitogen assay of fractions subjected to ion exchange chromatography. Fraction 0 is starting unfractionated CM, while Fraction 3 represents the bound material from anion exchange. Fractions 1 & 2 are unbound fractions from anion exchange. Fraction 4 (equivalent to Fraction 3) is the flow-through from the cation exchange column, while Fractions 5 & 6 represent the bound material. Cells were plated at 5000 cells per well, with all fractions used at 4%+20 ng/ml FGF-2. Cells were analysed 6 days after plating. Values represent proliferation relative to FGF-2 (set at 100%)+/−SD. n=6 for each set of conditions.

FIG. 11. Dose-response curve of greater than 30 kD neural stem cell mitogen (NSC-MF-1). Forebrain NSCs were plated at 5000 cells per well, with cells analysed 6 days after plating. Values represent proliferation relative to FGF-2 (set at 100%)+/SD. n=3 for each set of conditions.

FIG. 12. Dose-response curve of less than 10 kD neural stem cell mitogen (NSCMF-2). Forebrain NSCs were plated at 5000 cells per well, with cells analysed 6 days after plating. Values represent proliferation relative to FGF-2 (set at 100%)+/−SD. n=3 for each set of conditions.

FIG. 13. Comparison of mitogenic effects of NCS-MF-1 and -2. Forebrain NSCs were plated at 5000 cells per well, with cells analysed 6 days after plating. Values represent proliferation relative to FGF-2 (set at 100%)+/−SD. n=3 for each set of conditions

FIG. 14. Dose-response curve of NSC-MF-1 (>30 kD). Spinal cord neural stem cells were plated at 5000 cells per well, with cells analysed 8 days after plating. Values represent proliferation relative to FGF-2 (set at 100%)+/−SD. n=3 for each set of conditions.

FIG. 15. Dose-response curve of NSC-MF-2 (<10 kD). Spinal cord neural stem cells were plated at 5000 cells per well, with cells analysed 8 days after plating. Values represent proliferation relative to FGF-2 (set at 100%)+/−SD. n=3 for each set of conditions.

FIG. 16. Neural stem cells from the ventral mesencephalon display enhanced proliferation in NSC-MF-containing medium. Midbrain neural stem cells were plated at 2500 cells per well, with cells analysed 12 days after plating. Values represent proliferation relative to FGF-2 (set at 100%)+/−SD. n=3 for each set of conditions.

FIG. 17. MALDI MS profile of greater than 10 kD material (see methods for details of purification).

### EXAMPLES

Section A

### Example 1

### Preparation of NSC-MF

As disclosed herein, the conditioned medium of a human fibrosarcoma cell line, HT1080, comprises a neural stem cell mitogen.

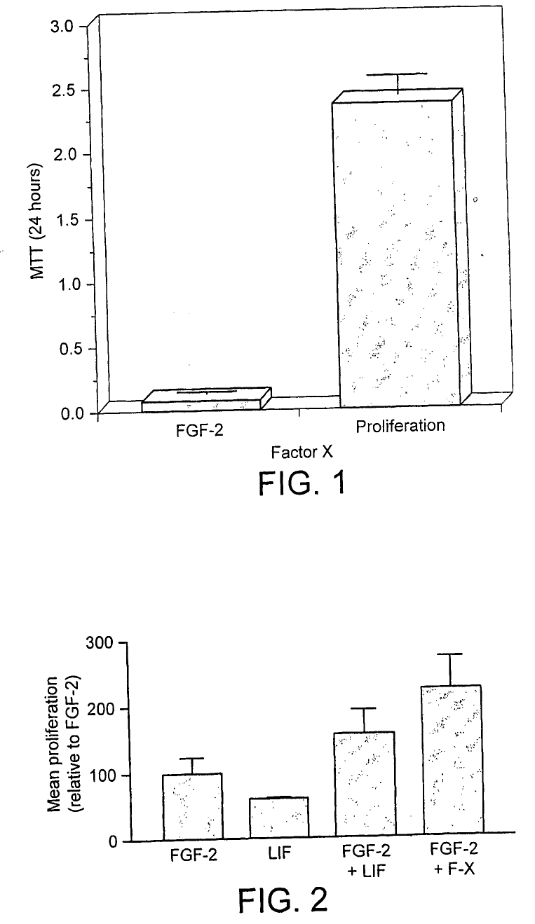

A 10% dilution of the conditioned medium from these cells, in combination with fibroblast growth factor-2 (FGF-2), increases the number of rodent forebrain neural stem/progenitor cells (NSCs) compared to FGF-2 alone (see FIG. 1).

A series of studies are performed to determine the identity of NSC-MF.

In this Example, NSC-MF is prepared in conditioned medium from HT1080 cells, which contains 10% foetal bovine serum.

Preparation of NSC-MF

HT1080 cells (ATCC #CCI-121) are grown in 225 cm² flasks in DMEM medium (Gibco) with 10% FCS (without antibiotics). When cells are approximately 90% confluent, the serum-containing medium is removed and the cells exposed to serum-free medium for 3648 hours. The medium is removed, centrifuged at 1000 g, and the supernatant collected from the pelleted cells. The medium is filtered through 0.22 μm filters and either used directly or frozen at −70° C. until needed.

It is found that the mitogenic activity (ie. NSC-MF) is secreted into the tissue culture medium, is sensitive to heating at 100° C. for five minutes, and is retained following filtration through a 0.22 μm filter. These characteristics are suggestive of a secreted protein.

To aid isolation of NSC-MF, the necessary conditions to propagate HT1080 cells under serum-free conditions are determined. Direct comparison between serum-free and serum-containing conditioned medium (combined with FGF-2) shows no significant differences in NSC proliferation, suggesting that NSC-MF is present at roughly identical concentrations in both sets of conditioned media. Studies outlined below are performed with serum-free conditioned medium (NSC-MF).

Thus, the novel neural stem cell mitogen (NSC-MF) disclosed herein is secreted by cells derived from a human tumour cell line. Conditioned medium from these cells prepared as above significantly increases the number of rodent forebrain neural stem/progenitor cells compared to the numbers obtained using fibroblast growth factor-2 (FGF-2), which is one of the most widely used commercially available neural stem cell mitogens.

The active CNS stem cell mitogen (NSC-MF) is present in conditioned medium from the tumour derived cell line as disclosed herein. NSC-MF is secreted into the medium and is sensitive to heat-inactivation, suggestive of a secreted protein. NSC-MF is also secreted into culture medium when the cells are exposed to serum-free medium. Direct comparison between serum-free and serum-containing conditioned medium shows no significant differences in neural stem cell proliferation, suggesting that NSC-MF is present in both sets of media, at roughly similar concentrations. This is very useful in the determination of further characteristics of NSC-MF. The further studies discussed hereinbelow use serum-free conditioned medium (NSC-MF).

Isolation and Characterisation of NSC-MF

NSC-MF is characterised using both HPLC and mass spectroscopy of serum-free conditioned culture medium.

Using HPLC, at least 9 major peaks are observed over a 70 minute time course.

Mass spectroscopy of the cell supernatant (This is an example of NSC-MF prepared as above) and a dialyzed sample reveals at least eight major protein peaks with approximate molecular weights of 8.7, 10.1, 11.8, 12.5, 13.6, 33.5 45.3 and 67.1 kilodaltons. This analysis is repeated at least three times with essentially identical results.

To purify NSC-MF to homogeneity, conditioned medium is freeze-dried, concentrated and subjected to HPLC with UV detection.

Approximately 200 mls of serum-free conditioned medium are freeze dried overnight. The residue is reconstituted in 5 ml of 0.05% v/v trifluoroacetic acid. The solution is filtered through a 0.45 μm filter and 4 ml of this solution is purified by HPLC.

HPLC conditions include a reverse phase C18 column, 5 m, 300 Å, 21×240 mm column. Buffer A is 0.05% v/v trifluoroacetic acid, Buffer B is 10% Buffer A+90% acetonitrile. The effluent is monitored by diode array (200 nm-400 nm), the flow rate is 7 ml/min. A linear gradient is used from 0% B to 90% B over 40 min. In this manner, a series of four continuous fractions are isolated, corresponding to approximately 7-15 minutes (Fraction 1), 15-28 minutes (Fraction 2), 2840 minutes (Fraction 3) and 40-50 minutes (Fraction 4) off the column. The fractions are collected, are freeze dried overnight and then re-constituted in water prior to use.

In this manner, a series of four continuous fractions are isolated, corresponding to approximately 7-15 minutes (Fraction 1), 15-28 minutes (Fraction 2), 2840 minutes (Fraction 3) and 40-50 minutes (Fraction 4) elution from the column (see FIG. 7 for HPLC profile).

These fractions are tested for mitogenic activity using our forebrain NSC proliferation assay. Forebrain NSCs are prepared as follows;

Cultures of fetal ventral forebrain cells are prepared as previously described (Minger S L, Fisher L J, Ray J, Gage F H. Long-term survival of transplanted basal forebrain cells following in vitro propagation with fibroblast growth factor-2. Experimental Neurology 1996 September; 141(1):12-24), with modification as set out herein. Briefly, the ventral forebrain ventricular zone including the septum pellucida is carefully dissected away from the adjacent lateral and medial ganglionic emineneces and surrounding cortical mantle from gestational day 13.5-14 (E14) Fischer 344 rat embryos (crown-rump length 9-11 mm). Tissue is collected in sterile Dulbecco's phosphate buffered saline (PBS), washed briefly with PBS, and then incubated in 0.1% trypsin/PBS for 30 minutes at 37° C. The tissue is subsequently washed, sedimented at 1000 g and resuspended in PBS-glucose three times and then dissociated to a single cell suspension by repeated pipetting through a series of narrowed Pasteur pipettes. Cell viability is determined by trypan blue exclusion and hemocytometric cell counting.

Forebrain cells are propagated under standard culture conditions in tissue culture flasks, multi-well plates (Coming/Costar) or 13 mm-dimater glass coverslips (BDH/Merck) as required, all precoated with 10 μg/ml polyomithine (Sigma) and 10 μg/ml laminin (Biogenesis). Cells are grown in DMEM/F12 high glucose medium containing N2 supplement (both from Life Sciences Technologies) and 20 ng/ml FGF2 (Chemicon), in 95% air/5% C02 humidified atmosphere. The medium is changed every three to four days and cells are passaged prior to confluency. Cells prepared in this manner are used for assays as described herein. The NSC proliferation assay is conducted as follows;

Cells are plated immediately after harvesting from brain at 5×10³ cells/well (2.63×1 cells/cm²) in 24 well plates, precoated with P+L, with factors added at the time of plating, with the medium completely changed every three days. Cells are monitored daily and cultures processed when cell density reaches 80-90% for any of the factors (usually 9-12 days after plating). The following factor(s) or mitogen(s) are used individually or in combination: heparin (0.05-2.0 μg/ml, Sigma), human fibroblast growth factor (FGF-2, 20 ng/ml, Chemicon), human leukemia inhibitory factor (LIF, 10-20 ng/ml, Chemicon), recombinant epidermal growth factor (EGF, 20 ng/ml, Chemicon), recombinant murine granulocyte macrophage colony stimulating factor (GM-CSF, 1-10 ng/ml, Preprotech), recombinant murine vascular endothelial cell growth factor (VEGF, 1-10 ng/ml, Preprotech), recombinant human insulin-like growth factor-1 (IGF-1, 10 ng/ml, Preprotech), recombinant human fibroblast growth factor-8 (FGF-8, 10 ng/ml, Preprotech), recombinant human thrombopoietin (100 ng/ml, Preprotech) and recombinant human neurotrophin-3 (10 ng/ml, Preprotech).

Six wells per condition are used for each experiment and the experiments are repeated at least twice. NSC-MF from crude serum-free conditioned medium is used at a 1:10 dilution with FGF-2-containing medium or at a 50% in N2-containing medium when used alone. Once one set of cells in each plate appears to be 80-90% confluent, 0.1 mg of MTT (3-[4,5-dimethylthiazol-2-yl]-2,5-diphenyltetrazolium bromide; Sigma, Chemical, Poole, UK) is added to each well. After a four-hour incubation at 37° C., the medium is removed, and 500 pl of dimethyl sulphoxide (DMSO, BDH-Merck, Lutterworth, and UK) is added to each well and vigorously mixed with a pipette. One hundred microliters from each well is transferred to a new 96-well plate and the absorbance at 630 nm is determined. Control blanks consist of the addition of all solutions without the cells. Each experiment uses cells grown in FGF-2 as the positive control and comparisons between various factors/mitogens is relative to that observed with FGF-2 alone.

To determine the relative mitogenic potency of various factors compared to NSC-MF, MTT assays are performed as above, using cells grown in the prescence/absence of other relevant factors/known mitogens. This is discussed in more detail in Example 2.

The HPLC fractions of NSC-MF (conditioned medium) are tested for mitogenic activity both combined with FGF-2 as well as alone.

The fraction(s) containing mitogenic activity are subjected to further purification and mass spectroscopy analysis until NSC-MF is purified to homogeneity.

NSC-MF is purified from serum-free conditioned culture medium using HPLC and mass spectroscopy. Using HPLC, 9 major peaks are observed.

Mass spectroscopy of the crude supernatant and a dialyzed sample reveals at least eight major protein peaks with approximate molecular weights of 8.7, 10.1, 11.8, 12.5, 13.6, 33.5 45.3 and 67.1 kilodaltons. This analysis is repeated three times with essentially identical results obtained each time.

A continuous series of fractions obtained by time intervals across the HPLC gradient is analysed for mitogenic activity (both alone and with FGF-2). This analysis allows attention to be focussed on a smaller number of candidate proteins, facilitating the identification of mitogen(s) in the conditioned medium (ie. NSC-MF and/or any other or related mitogen(s)).

Thus, NSC-MF is a novel neural stem cell mitogenic factor.

NSC-MF is identified by the properties set forth in this disclosure.

The particular (eg. molecular) identity of NSC-MF is determined by the methods set out above and in the following Examples.

### Example 2

### Use of NSC-MF

As shown herein, NSC-MF is useful as a mitogen. It is further disclosed herein that NSC-MF is useful in the potentiation of other mitogenic signals.

Use of NSC-MF in combination with known mitogens

To demonstrate that the mitogenic activity in the HT1080 conditioned medium is due to NSC-MF rather than to a previously identified NSC mitogen, direct comparisons between NSC-MF and several commercially available mitogens are performed. The standard conditions for the long-term propagation of forebrain NSCs (see above) are used throughout this Example, including the propagation of NSCs on a defined substrate of poly-omithine and laminin (P+L), the use of serum-free, DMEM/F12 medium containing N2 neuronal supplement, and 20 ng/ml FGF-2. Under these conditions, forebrain NSCs proliferate for at least five months, continue to generate neuronal cells throughout this time period, and are susceptible to retroviral-mediated genetic modification and long-term transgene expression.

Freshly dissected forebrain NSCs are plated at low density (5×10³ cells per well) in 24-well plates and the mitogens added at the time of plating (6 wells per mitogen/condition). Forebrain NSCs exposed to 20 ng/ml FGF-2 alone are used as the positive control in each set of experiments and mitogenic activity is expressed relative to the baseline of FGF-2 alone.

Commercially available mitogens are used over a range of concentrations according to the published/recommended concentration for NSC or other stem cell cultures. Serum-free conditioned medium (NSC-MF) is used at a 1:10 dilution when added to other factors and at a 50% dilution when used alone.

To exclude the possibility that the activity of NSC-MF might merely represent additional FGF-2 secreted into the conditioned medium, forebrain NSCs are propagated in increasing concentrations of FGF-2 (20, 50, 100 and 200 ng/ml); no significant increase in NSC cell number is observed with FGF-2 concentrations above 20 ng/ml, showing that NSC-MF is not FGF-2.

Epidermal growth factor (EGF) has also been shown to be a potent NSC mitogen. Although 20 ng/ml EGF is relatively potent when forebrain NSCs are grown in suspension as spheres, forebrain NSCs survive very poorly in EGF when grown on P+L; increasing the concentration of EGF up to 100 ng/ml is ineffective in promoting NSC proliferation.

These data show that NSC-MF is distinct from FGF-2 and EGF, the most potent prior art NSC mitogens available.

Heparin and heparin sulfate are compounds known to potentiate the mitogenic effects of FGF-2 on NSCs. No significant increase cell number is observed when forebrain NSCs are propagated with 20 ng/ml FGF-2 and a range of heparin concentrations (0.05-2.0 μg/ml). Therefore, NSC-MF is not heparin or heparin sulfate.

To determine if NSC-MF is identical with other mitogen(s) which induce proliferation of stem cell populations, forebrain NSCs are propagated in granulocyte-macrophage colony stimulatory factor (GM-CSF, 1-10 ng/ml) and vascular endothelial growth factor (VEGF; 10 ng/ml). Neither of these factors promotes NSC proliferation; doubling the initial number of cells plated at the beginning of the experiment to 1×10⁴ had no significant effect with either factor. Thus, NSC-MF is not GM-CSF, and is not VEGF. Furthermore, NSC-MF exhibits significant NSC mitogenic effect, in contrast to these prior art stem cell mitogens.

In addition to the well-characterised mitogenic effects of FGF-2 and EGF on various NSC populations, it has also been recently shown that the combination of 10 ng/ml LIF and 20 ng/ml FGF-2 is a potent mitogenic cocktail for human fetal forebrain NSC neurospheres, resulting in significantly greater proliferation than FGF-2 alone (see Carpenter M K, Cui X, Hu Z Y, Jackson J, Sherman S, Seiger A, Wahlberg L U. In vitro expansion of a multipotent population of human neural progenitor cells. Experimental Neurology 1999 August; 158(2):265-78). It is therefore determined whether this combination of mitogens would be more effective than FGF-2 using the NSC culture conditions as set out above. The combination of LIF+FGF significantly enhances forebrain NSC proliferation compared to 20 ng/ml FGF alone (See FIG. 2, mean 60% increase in cell number after 9 days of treatment; p<0.01), whereas 10 or 20 mg/ml LIF alone is markedly less effective than 20 ng/ml FGF-2 (see FIG. 2).

The combination of NSC-MF+20 ng/ml FGF-2 is significantly more effective than FGF+LIF, resulting in more than a 40% increase in NSC cell number compared to LIF+FGF (See FIG. 2, p≦0.017). It is shown that forebrain NSCs plated at low density and propagated in 50% NSC-MF (serum-free conditioned medium) without added mitogens proliferate for at least nine days (the duration of this particular experiment), at approximately 50% the efficiency observed with FGF-2, demonstrating that NSC-MF has mitogenic activity even in the absence of FGF-2.

In addition to the potentiation of NSC proliferation with FGF-2, NSC-MF also potentiates the mitogenic effects of both LIF and LIF+FGF-2, increasing NSC cell number compared to that obtained with either set of factors alone. This shows that NSC-MF potentiates the effects of a variety of stem cell mitogen(s) and stem cell populations.

NSC-MF has NSC survival-promoting effects. Without wishing to be bound by theory, these effects may be mechanistically independent of its mitogenic effects. NSCs plated a very low densities (250-1000 cells per well) are capable of survival and subsequent expansion when plated in the presence of NSC-MF+FGF-2; similar cells plated in FGF-2 or any of the other mitogens alone do not survive.

NSC-MF is screened against known stem cell mitogens as above. NSC-MF is neither FGF-2 nor epidermal growth factor (EGF), the most potent neural stem cell mitogens currently commercially available. NSC-MF is not recombinant human insulin-like growth factor-1 (IGF-1, 10 ng/ml, Preprotech), recombinant human fibroblast growth factor-8 (FGF-8, 10 ng/ml, Preprotech), recombinant human thrombopoietin (100 ng/ml, Preprotech) or recombinant human neurotrophin-3 (10 ng/ml, Preprotech). 20 ng/ml EGF is a very poor mitogen for forebrain neural stem cells, and 20 ng/ml FGF-2 results in maximal proliferation; simply increasing the concentration of FGF-2 does not significantly increase stem cell number. NSC-MF is, however, synergistic with a potentiator of the effect(s) of FGF-2; significantly greater neural stem cell proliferation is observed with a combination of NSC-MF (10% conditioned serum-free medium) and 20 ng/ml FGF-2 compared to an identical concentration of FGF-2 alone (mean 93% increase in cell number with NSC-MF+FGF after 15 days; See FIG. 1). Similar to EGF, leukemia inhibitory factor (LIF, 10-20 ng/ml), granulocyte-macrophage colony stimulatory factor (GM-CSF, 1-10 ng/ml), and vascular endothelial growth factor (VEGF; 10 ng/ml) are poor mitogens for forebrain neural stem cells when used alone, and hence do not resemble NSC-MF.

The combination of 10 ng/ml LIF with 20 ng/ml FGF-2 is a potent mitogenic cocktail for human fetal forebrain neural stem cells, and results in significantly greater stem cell proliferation than FGF alone (Carpenter et al, 1999—ibid). The combination of LIF+FGF also results in significantly enhanced proliferation of rodent forebrain stem cells (mean 60% increase in cell number after 9 days of treatment compared to 20 ng/ml FGF alone, See FIG. 2).

NSC-MF+FGF-2 is significantly more potent in inducing neural stem cell proliferation than the combination of LIF+FGF (mean 43% increase in cell number after 9 days compared to LIF+FGF, See FIG. 2). Thus, the conditioned culture medium of the invention contains a factor (ie. NSC-MF) that represents a potent neural stem cell mitogen.

The utility of NSC-MF as a mitogen is clearly demostrated.

Further uses of NSC-MF are described herein, for example the use of NSC-MF in cell expansion applications.

NSC-MF Potentiates Cell Population Expansion

Longer-term timecourse studies are performed to determine the extent to which NSCs remain sensitive to the effects of NSC-MF and whether specific (sub)populations of cells are particularly affected.

Freshly harvested forebrain stem/progenitor cells are plated at an initial density of 1×10⁵ cells in P+L 75 cm² flasks (1.33×10³ cells/cm²) and propagated in either FGF-2 alone or 10% conditioned medium (NSC-MF) in FGF-2-containing medium (7.5 mls of medium per flask) until approximately 75% confluent. At that time, cells are trypsinized off the substrate, collected and washed. The total number of cells recovered from each flask is determined, and 1×10⁵ cells is added to a new 75 cm² flask and propagated until 75% confluent as described above. At each passage, cells are also plated onto coverslips for immunocytochemical assessment. The total expansion in cell number is determined from the original 100,00 cells.

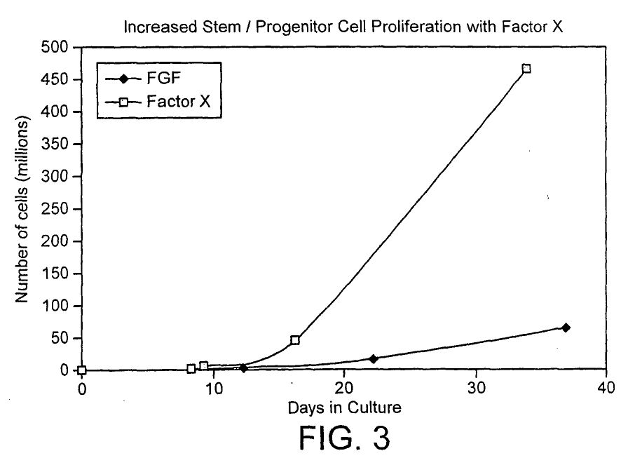

NSC-MF's effects are robust for at least 40 days after CNS stem cells are isolated from the brain with a greater than 4000-fold expansion of neural stem cells over this time course, using a 1:10 dilution of conditioned medium (NSC-MF) and 20 ng/ml FGF-2. Using cells grown under identical conditions in FGF-2 alone, NSC-MF+FGF2 treatment results in an approximately 10-fold expansion in neural stem cell number compared to FGF alone treatment over this time course (see FIG. 3).

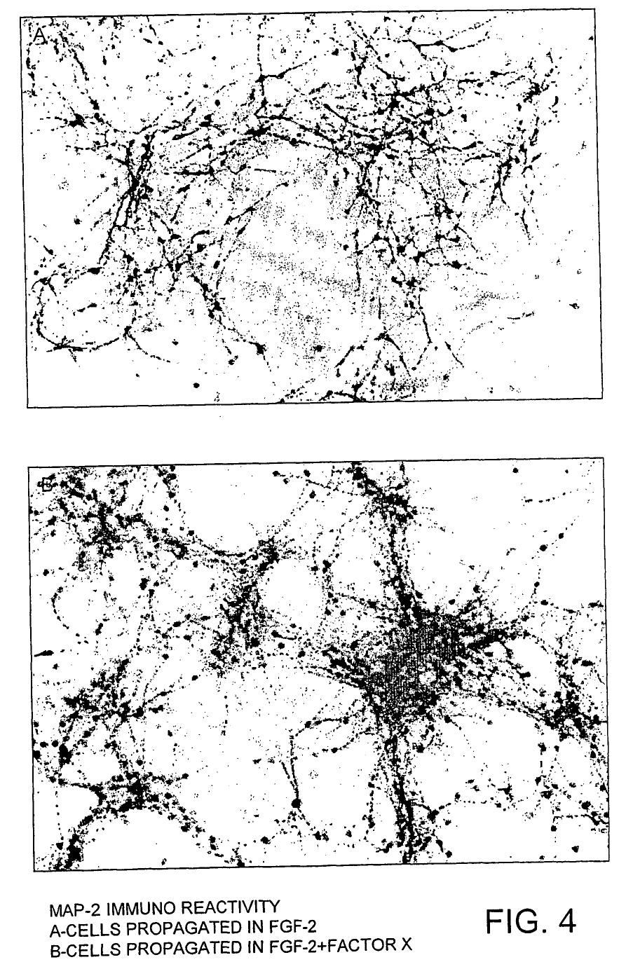

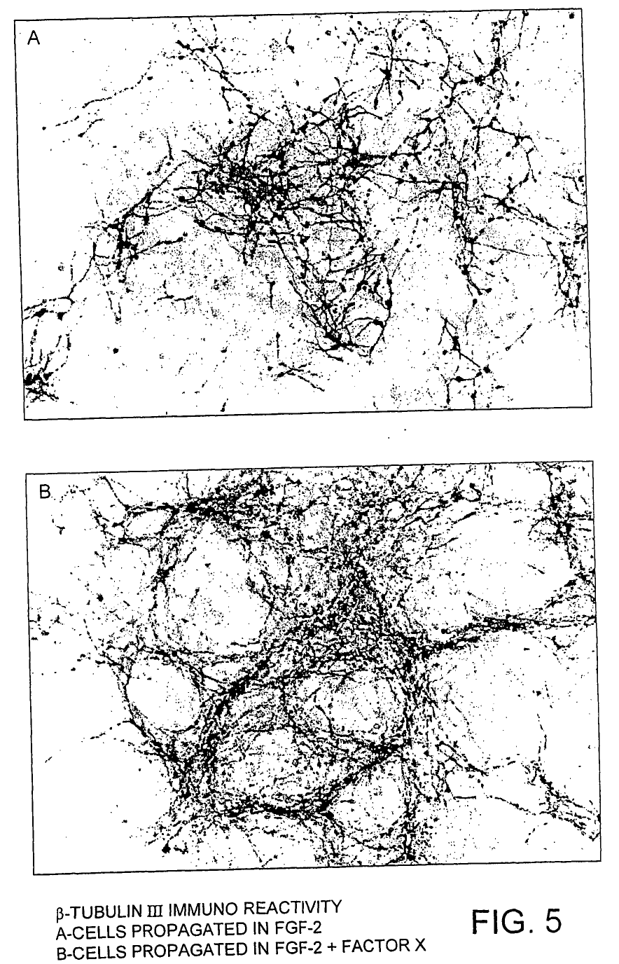

A direct comparison between neural stem cells grown in FGF-2 and those grown in NSC-MF+FGF-2 shows that, in addition to significantly increasing total neural stem cell number over time, cells grown in NSC-MF yield greatly increased numbers of differentiated neurons (as revealed by positive MAP-2 and β-tubulin immunoreactivity). Quantitative analysis shows that there is an approximately 4-fold enrichment in neurons compared to the number of neurons derived from cells cultured in FGF-2 alone (see FIGS. 4 and 5). The neurotransmitter phenotype of these cells is examined to determine which population(s) of neuronal progenitor cells are most susceptible to this useful property of NSC-MF.

Use of NSC-MF in Propagation of Cholinergic Neuronal Progenitor Cells

Expansion of neurotransmitter-specific neuronal populations is a problem of neural stem cell research.

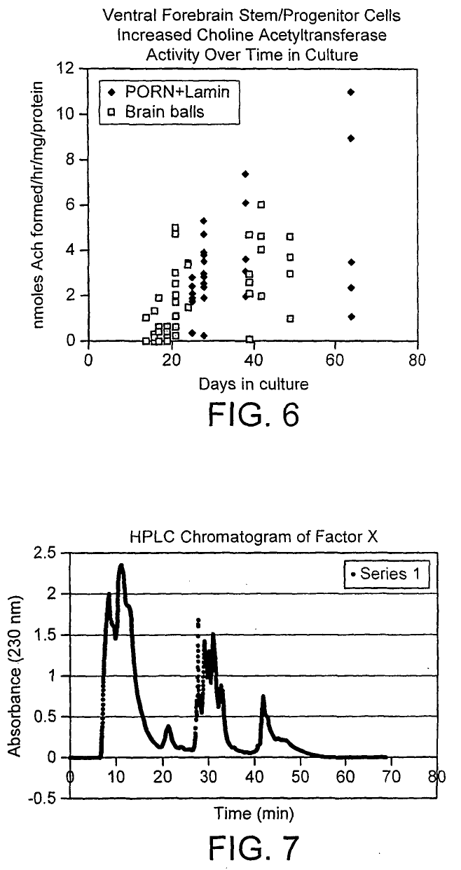

Rodent ventral forebrain progenitor cells rarely generate neurons that synthesise acetylcholine during the first three weeks of expansion in FGF-2 (see FIG. 6).

Stem/progenitor cells are analysed using immunocytochemistry. Proliferating neural stem/progenitor cells are harvested at various time points from cultures and plated onto 13 mm-diameter glass coverslips precoated with P+L in 24-well plates at the density of 5×10³ cells/well. 48-72 hours later, the cells are induced to differentiate by withdrawal of FGF-2 and substitution with medium containing 100 ng/ml all-trans retinoic acid (Sigma), 1 ng/ml FGF-2, 100 ng/ml nerve growth factor (NGF) or 1% fetal calf serum (constituents used individually or in combination). The medium is changed every three days, and the cells analyzed 6-12 days after the onset of differentiation. To determine the cellular phenotype of propagated cells, the coverslips are gently washed with PBS, fixed for 30 minutes with 4% paraformaldehyde, washed twice with PBS and then placed into 0.1% Triton X-100 in Tris-buffered saline (TBS+) for 30 minutes at room temperature. Nonspecific antibody binding to the cells is blocked by incubating the cells with 5% milk and 0.1% Triton X-100 in Tris-buffered saline (TBS+) for 30 minutes at room temperature prior to an overnight incubation at 4° C. with primary antibody diluted in TBS+/5% milk. The following day, the primary antibody is removed and cells are washed three times for 15 minutes each in TBS+, and then incubated with species-specific horseradish peroxidase-conjugated secondary antibody (Jackson Labs) for one hour at room temperature. Following three 15 minutes washes in TBS, the cells are exposed to a 0.05% solution of diaminobenzidine, 0.04% nickel chloride and 0.01% H₂O₂ for 6-15 minutes. The coverslips are then briefly dehydrated through a series of graded alcohols and mounted on glass microscope slides and analyzed. Optimal antibody concentration for each individual antibody is determined using dilution curves with primary neurons, and the specificity of antibody binding is confirmed by omission of primary antibodies as controls in each immunocytochemical procedure.

A broad panel of primary antibodies specific for a variety of neuronal and non-neuronal antigens are used to characterize cells in the cultures, including nestin (Chemicon), a marker for neuroepithelium-derived progenitor cells; β-tubulin III (Sigma), NeuN (Chemicon), and microtubule-associated protein (MAP-2; Roche; UK) which recognize differentiated neuronal cells; gamma amino butyric acid (GAD; Chemicon) specifically for GABAergic neurons; low affinity nerve growth factor receptor (p75; Roche), tyrosine receptor kinase-A (TrkA; Santa Cruz Antibodies) and choline acetyltransferase (CHAT; Chemicon) antibodies for identification of cholinergic neurons, with glial acidic fibrillary protein (GFAP; Dako) and anti-galactocerebroside (GalC, Roche) and −O4 (Roche) antibodies used to identify astrocytes and oligodendrocytes, respectively.

Nerve growth factor (NGF) survival assays are performed. To determine the relative number of cholinergic progenitor cells grown under various conditions and factors, ventral forebrain stem/progenitors cells assayed for choline acetyltransferase (ChAT) activity. Briefly, forebrain cells are plated at approximately 5×10⁴ cells/well in 6-well plates and propagated in mitogen-containing medium until the cells are approximately 90% confluent, at which time the mitogen is withdrawn and 100-200 ng/ml NGF (Chemicon) in DMEM/F12/N2 is added. The NGF-containing medium is replaced every three-four days and each well is monitored daily for cell survival after initial exposure to NGF. In cases where cells did not survive long-term in NGF alone, the last day surviving cells are observed is recorded.

ChAT enzymatic assays are conducted based on the technique of Fonnum (1976) as previously described for cultured neurons (Minger, 1996—ibid). Cells are harvested on ice 14-28 days after exposure to NGF using 0.1% trypsin and then homogenized in 100 μl of a solution containing 0.87 mm EDTA and 0.1% Triton X-100, pH 0.7.0. Ten μl of cell homogenates is incubated at 37° C. for one hour in 10 μl of a mixture of 50 μM sodium phosphate, 300 mM sodium chloride, 9 mM EDTA, 2 mM choline chloride, 100 μM eserine salicylate, 0.5 mg/ml bovine serum albumin and 100 μM [acetyl-1¹⁴C]-coenzyme A. The reaction is halted by adding 100 μl ice cold distilled water. One ml of extraction buffer (0.5% sodium tetraphenylboron in 85% toluene/15% acetonitrile) is then added to each sample and samples are centrifuged at 15,000 rpm for two min. A 650 μl aliquot of the supernatant is removed, mixed with 5 ml Ecoscint scintillation fluid and the radioactivity incorporated into the product is counted with a beta-counter for ten minutes. Controls consisted of the incubation and extraction solutions without cells. Each sample is assayed in triplicate and the average blank counts are subtracted from the sample counts prior to correcting for protein. Protein concentrations are assessed in duplicate using Comassie Plus protein assay reagent (Pierce) and measuring absorbance at 595 nm. Bovine serum albumin is used as a protein standard. Data are expressed as nanomoles of acetylcholine (ACh) formed per hour per mg protein or per well.

A significant increase in the activity of the synthetic enzyme choline acetyltransferase (ChAT; see FIG. 6), as well as the number of p75- and ChAT-immunoreactive neurons is observed in cultures beginning approximately three-four weeks after isolation from the brain and subsequent exposure to 100 ng/ml nerve growth factor. After four to six weeks of expansion, the number of cholinergic neurons in FGF-2 expanded cultures increases from less than 5% of the total neuronal population to approximately 25% depending on the culture conditions.

Analysis of cells cultured in NSC-MF+FGF-2 shows that significantly increased numbers of cholinergic neuronal progenitor cells are generated compared to NSCs propagated in FGF-2 alone over the same timecourse.

Without wishing to be bound by theory, these useful effects of NSC-MF may be brought about by NSC-MF promoting the expansion of a more pluripotent NSC, or by

NSC-MF having specific effect(s) on cholinergic progenitor cells, or by a different mechanism.

NSC-MF is useful in propagation of different neuronal cell type(s).

### Example 3

### Properties of NSC-MF

Further physico-chemical and proliferative properties of NSC-MF according to the present invention are demonstrated.

Serum-free conditioned medium is collected from the HT1080 cells and frozen at −80° C. The medium is thawed on ice and then dialysed with PBS buffer using dialysis tubing with molecular weight cut-offs of either 1000 Da or 3500 Da. Afterwards, the dialysed samples are filtered through a 0.22 μm syringe filter and assessed for mitogenic activity using a proliferation assay as discussed above (Briefly: 5×10³ neural stem cells/well; 11-12 wells per condition; medium completely replaced on days 4 and 7, with cells analysed by MTT on day 8; procedure as described in Example 1).

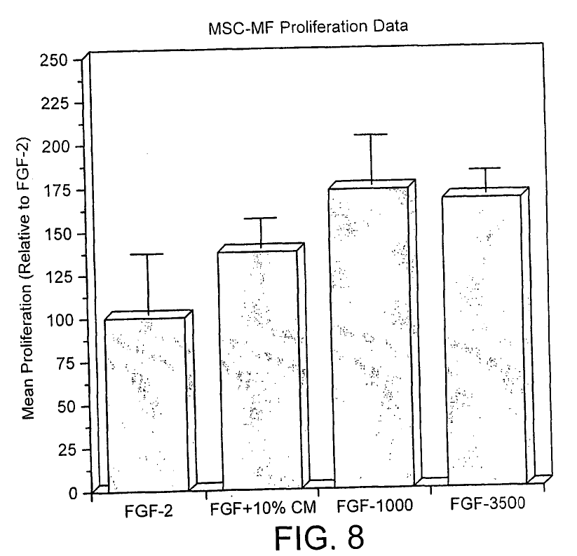

The graph shown in FIG. 8 indicates relative proliferation of cells grown in

a) 20 ng/ml FGF-2 alone (column 1)

b) 10% serum-free conditioned medium (ie NSC-MF)+20 ng/ml FGF-2 (column 2)

c) 10% concentration of dialysed NSC-MF conditioned medium with 1000 Da cutoff+20 ng/ml FGF-2 (column 3)

d) 10% concentration of dialysed NSC-MF conditioned medium with 3500 Da cutoff+20 ng/ml FGF-2 (column 4)

The raw data has been normalised to that obtained with FGF-2, with the mean OD for FGF-2 set at 100% (as in Example 2). Using ANOVA and post-hoc analysis, all three NSC-MF conditions are significantly more potent than FGF-2 alone, and both the dialysed samples result in significantly greater proliferation than that seen with 10% conditioned medium (p<0.05).

Thus, proliferative properties of conditioned medium NSC-MF and fractions thereof are demonstrated to be potent mitogens according to the present invention.

### Example 4

### NSC-MF Selectively Potentiates the Proliferation of Neuronal Progentor Cells

Molecular markers characteristic of neural progenitor cells are known in the art and include MAP-2 and β-tubulin III. In this Example, the ability of NSC-MF to selectively potentiate the proliferation of neural progenitor cells is demonstrated using a population of freshly dissected forebrain NSCs (see previous Examples) treated as described below.

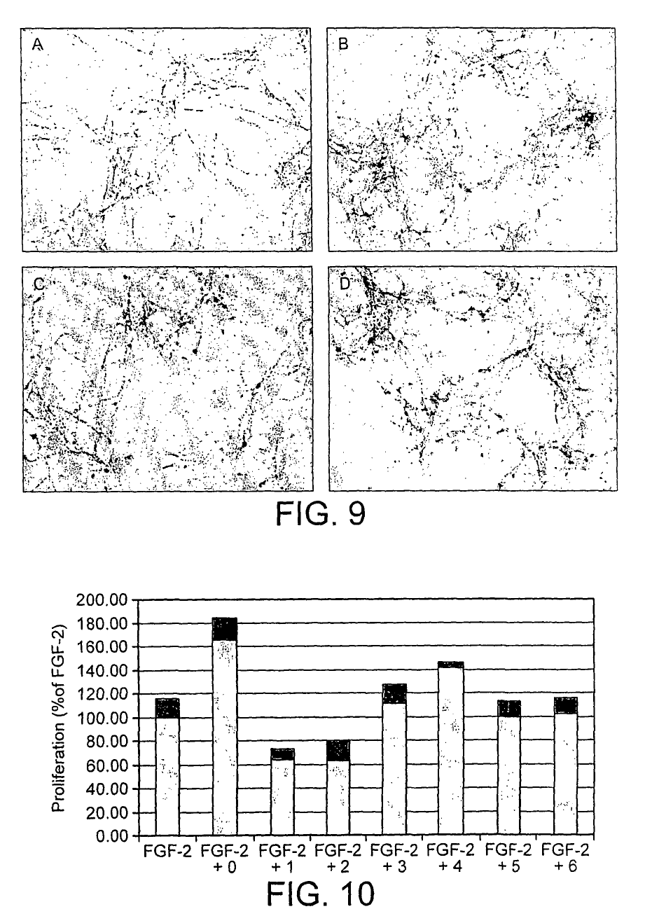

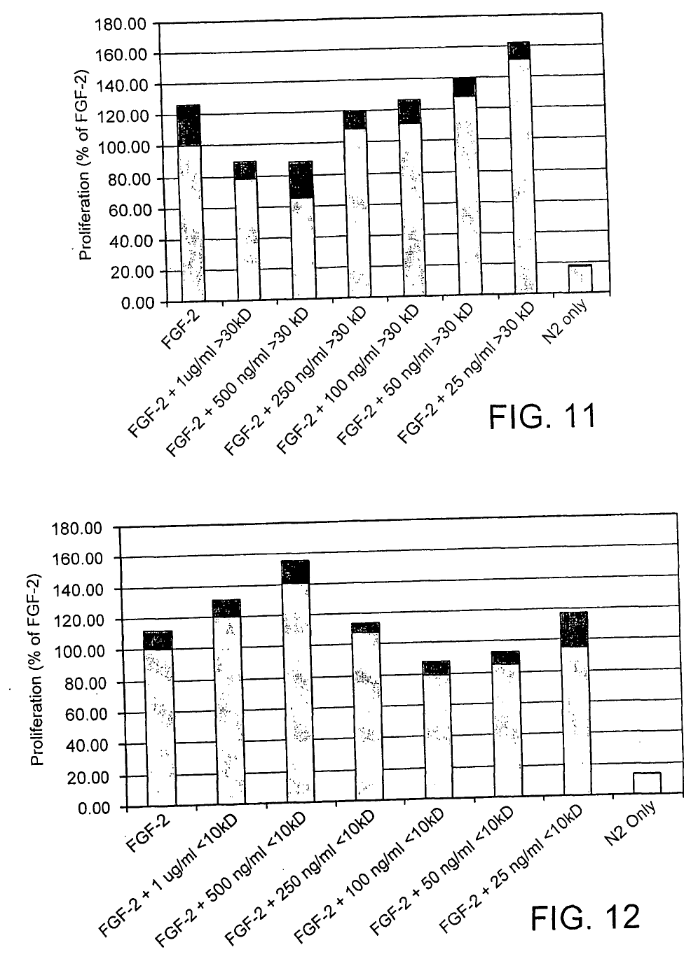

The cells displayed in the photomicrograph of FIG. 9 are identical cells propagated under identical culture conditions, with the exception that the NSCs in panels a and c are propagated in 20 ng/ml FGF-2 alone, whilst those in b and d are grown in 10% serum-free conditioned medium (NSC-MF)+20 ng/ml FGF-2. Both sets of cells are expanded for two weeks, placed at 5×10³ cells/well on P+L coverslips, differentiated for 6 days in differentiation medium and them processed for immunocytochemistry (as described in Example 2).

Panels a and b of FIG. 9 show cells stained for the neuronal marker β-tubulin III, whilst those in panels c and d show positive immunoreactivity for microtubule-associated protein-2 (MAP-2), another neuronal marker. (See also FIGS. 4 and 5)

Thus, it is demonstrated that NSC-MF according to the present invention selectively potentiates the proliferation of neural progenitor cells.

Section B

Methods

Biochemical purification. The HT1080 conditioned medium (approximately two litres) was dialysed using a 3.5 KDa molecular weight cut off membrane using phosphate buffered saline. The dialysis sacks were then placed in polyethylene glycol 20000 to concentrate the material to approximately 50 ml. The concentrate was affinity purified using a lectin-Sepharose column 4B column (Amersham-Pharmacia) equilibrated with 25 mM HEPES (pH 6.5). The activity was retained in the flow through and elution with 250 mM alpha-methyl mannoside failed to yield the active component active component. In subsequent procedures the affinity purification step was omitted.

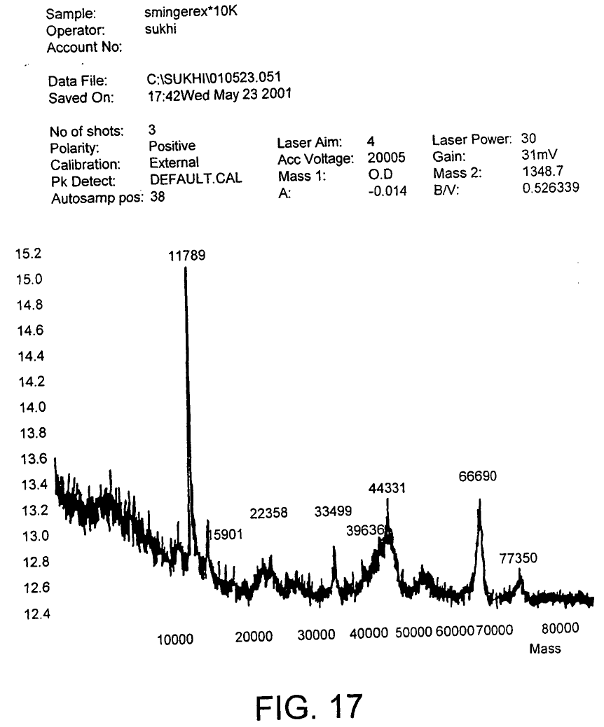

The concentrate was then fractionated with a 30 KDa cut-off centrifugal filter, Biomax 5, Millipore. The activity was retained in both of these fractions. The greater than 30 KDa fraction was purified by anion exchange chromatography using a HITRAP Q Sepharose (Amersham-Pharmacia) using 20 mM Tris hydrochloride buffer (pH 8). The active component was retained on the column and was eluted with a gradient of 1 M sodium chloride. Cation exchange chromatography on a HI TRAP SP column with phosphate buffer pH 6.8 was carried out with the active component not being absorbed onto the column. The fraction was analysed by matrix assisted laser desorption mass spectrometry (ThermoBioanalysis) using saturated sinapinic acid as the matrix (see FIG. 17). The fraction was also analysed by native gel electrophoresis, 7.5% resolving gel using Tris Glycine buffer, with five bands obtained on staining with Coomassie blue. In parallel experiments unstained bands have been excised and then electroeluted with 50 mM ammonium bicarbonate buffer.

The Coomassie blue stained bands were excised and placed in acetonitrile/50 mM ammonium bicarbonate (60:40, 250 μl) and then gently agitated on a shaker for 30 min. This process was repeated and then the bands were shaken in acetonitrile 120 mM ammonium bicarbonate (60:40, 250 μl). The gel bands were completely dried in a speedeVac™ dryness. The dried gel bands were treated with trypsin (0.5 μg/mm³ in 20 mM ammonium bicarbonate buffer for 15 min when the gel absorbed the trypsin, 10 μl of 20 mM ammonium bicarbonate was added and the reaction was incubated at 37° C. for 24 h. The digest (1 μl) was placed on the MALDI target followed by the matrix and the samples were analysed by MALDI MS. The resultant fragments for three of the five bands were then compared to fragments in the databases, where no significant matches to known proteins were obtained.

Mitogenic assays. In accordance with Section A, the assays examining mitogenic activity of NSC-MF are based on MTT (mitochondrial respiration) assays. Neural stem cells were plated immediately after dissection from foetal brain in 24-well plates containing poly-ornithine and laminin substrates (each at 10 μg/ml) at densities ranging from 2500-10,000 cells/well, depending on the neuroanatomical source of the cells and the experimental conditions. Unless otherwise stated, assays were performed with cells derived from embryonic day 14 forebrain. In each individual experiment, cell density at time of plating was identical across all conditions. Mitogens were added at the time of plating with the medium completely changed every three days. Cells were processed for MTT when one or more sets of cells were 80-90% confluent, usually 6-12 days after plating. For most experiments, the positive control and reference standard were neural stem cells grown in the presence of 20/mg fibroblast growth factor-2 (FGF-2) alone, the standard neural stem cell mitogen in use in our lab. Under these conditions, cellular proliferation was compared to FGF2 alone, and the data expressed as the percentage of proliferation compared to FGF2+/−standard deviation.

Discussion

As reported above, the neural stem cell mitogenic activity secreted by HT1080 cell line was heat labile, could be concentrated by dialysis and/or centrafiltration, and was greater than 3.5 kilodaltons (kD) in size. In assays directly comparing the mitogenic activity of the mitogen of the present invention to those of other known factors, we found that the mitogen of the present invention is not any one of: leukaemia inhibitory factor (LIF), epidermal growth factor (EGF), sonic hedgehog protein (SHH), fibroblast growth factors-2 and -8 (FGF-2, FGF-8), nerve growth factor (NGF), vascular epithelial growth factor (VEGF), stem cell factor (SCF), granulocyte-macrophage colony stimulating factor (GM-CSF), thromopoietin, insulin-like growth factor-1 (IGF-1), platelet-derived growth factor (PDGF) and hepatocyte growth factor (HGF) as potential candidate mitogens. Thus, we conclude that the neural stem cell mitogen obtainable from the HT1080 conditioned medium represents a novel stem cell mitogen.

In our initial experiments, conditioned medium (CM) was subjected to gel filtration chromatography using a Superdex 75 column. Three very large protein peaks were obtained. 14 individual fractions were collected across the three peaks with each one assessed for mitogenic activity using the standard NSC proliferation assay with 20 ng/ml FGF-2 and 4% dialysed CM (NSC-MF)+20 ng/ml FGF-2 as positive controls. None of the 14 individual fractions contained demonstrable mitogenic activity. We then collected three fractions each of which spanned one entire protein peak, but again no significant mitogenic activity was found compared to the potent NSC proliferation induced by the dialyzed sample. These findings suggested that the NSCMF protein was not a significant component of the CM, but rather represented a very minor fraction of the total secreted protein. Due to the low abundance of the NSC-MF protein within the CM sample, large quantities of CM were required to facilitate the isolation and purification of NSC-MF and upwards of six litres of conditioned medium were collected.

Taupin et al ((2000) Neuron, vol 28, pp 385-397) have reported on the isolation and characterisation of cystatin C. Cystatin C potentiates the mitogenic effects of FGF-2 on adult neural stem cells. In our studies, we investigated whether NSC-MF was cystatin C. Utilising a Sepharose 4B heparin-binding column to which cystatin C and other heparin-like factors selectively bind, we discovered that NSC-MF did not bind to the column, but rather was found in the unbound fraction that passed through the column when bound and unbound material were tested for mitogenic activity using our standard assay. This finding not only demonstrated that NSC-MF was distinct from cystatin C and other heparin-like factors (and therefore may not be a glycolsylated protein), but also the use of the Sepharose 48 column surprisingly provided us with a means of partially purifying NSC-MF away from a number of other proteins in the CM which bind to the 4B column.

Using CM that had been partially purified by Sepharose 4B column chromatography, the unbound fraction containing NSC-MF was then subjected to anion and cation exchange chromatography. Both bound and unbound fractions were obtained from each typ of column and subjected to our standard mitogenic assay. As can be seen in FIG. 10 below, NSC-MF binds selectively to an anion exchange column, allowing for further purification from proteins that pass through the column.

It is clear from FIG. 10 that most of the mitogenic activity can be recovered from column-bound Fraction 3. The use of sequential Sepharose 4B and anion exchange chromatographic techniques therefore, represents an efficient means of partially purifying NSC-MF from other contaminating proteins. This procedure also has uncovered other potentially interesting species from HT1080 CM. For example, in FIG. 10 above, it can be observed that proteins in both Fractions 1 & 2 have very potent anti-proliferative effects, suppressing the mitogenic effects of FGF-2 by approximately 40%.

Using the purification scheme as outlined above, we initiated a series of experiments to determine the approximate molecular weight of NSC-MF. Conditioned medium subjected to ion exchange chromatography was fractionated by molecular weight into three sets of proteins: less than 10 kd, 10-30 kD and greater than 30 kD. Rigorous investigation of the three different protein fractions (>30 kD, 10-30 kD, and <10 kD) revealed that there may be at least two distinct neural stem cell mitogens in the HT1080 conditioned medium, one which has a molecular weight greater than 30 kD and one which is less than 10 kD. These at least two neural stem cell mitogens will be referred to collectively as neural stem cell-mitogenic factors (NSC-MFs)—but are also designated NSC-MF-1 (>30 kD) and NSC-MF-2 (<10 kD) for convenience.

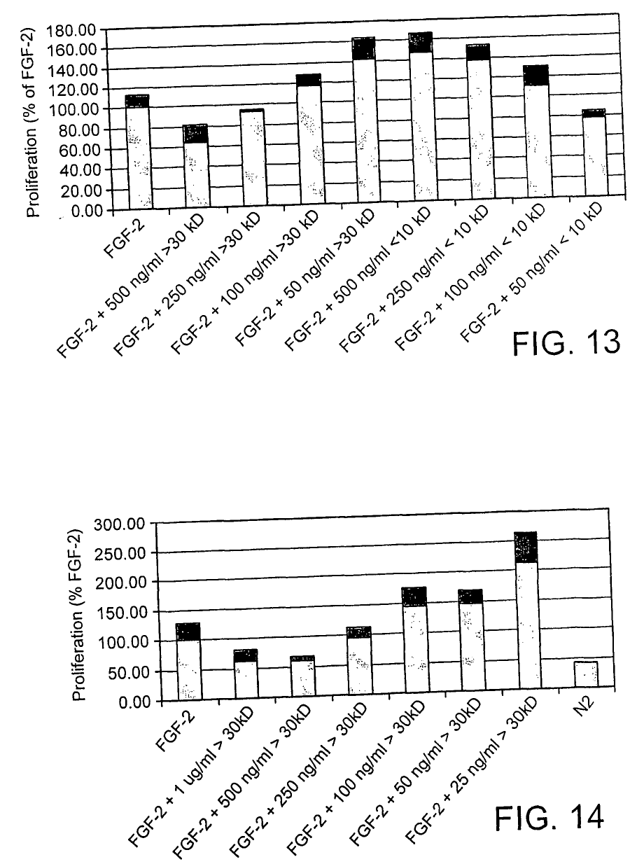

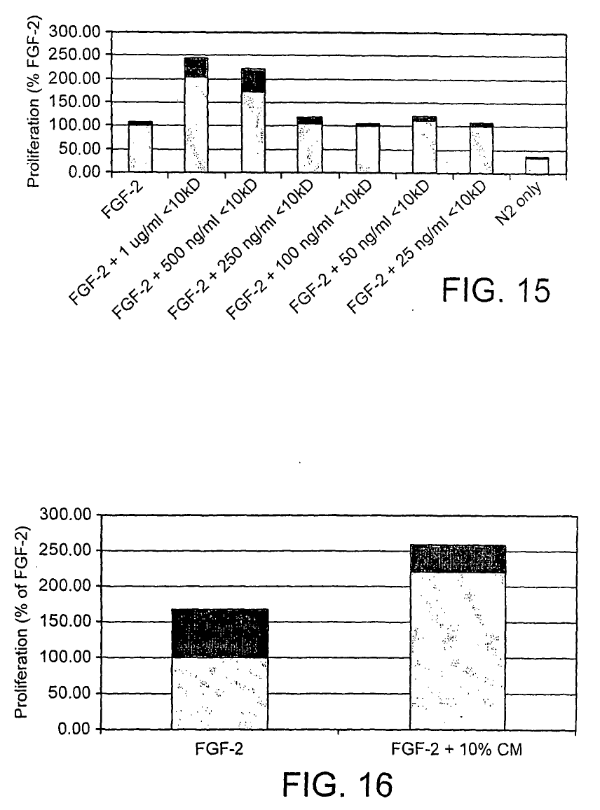

As can be seen in FIGS. 10 and 12 below, each of the mitogens displays a distinct concentration-dependent mitogenic profile, but has a similar overall effect on forebrain neural stem cell proliferation (approximately 40-50% increase in proliferation each compared to FGF-2 alone) when directly compared to one another (FIG. 13).

Preliminary analysis reveals that when combined, the factors may have additive and perhaps synergistic effects on forebrain neural stem cell proliferation.

In addition to neural stem cells derived from the embryonic forebrain, we have also shown that neural stem cells derived from the gestational day-14 spinal cord are sensitive to the mitogenic effects of both the greater than 30 (NSC-MF-1) and less than 10 kD (NSC-MF-2) neural stem cell mitogens, with similar concentration effects to that observed for the forebrain neural stem cells. Spinal cord stem cells were obtained from the gestational day 14 spinal cord at approximately C8-T4 level, and cultured using identical methods as described for forebrain neural stem cells (Minger et al, 1996—ibid).

### SUMMARY

A novel mitogen is obtainable from the HT1080 cell line conditioned medium.

The neural stem cell mitogenic activity represented by NSC-MF in the HT1080 cell line conditioned medium may be attributable to two distinct mitogens, one of greater than 30 kilodaltons (kD) and one that is less than 10 kD.

The mitogen(s) of the present invention significantly increase embryonic rodent forebrain neural stem cell proliferation by approximately 40-50% when combined with FGF-2 compared to FGF-2 alone.

The mitogen(s) of the present invention significantly increase the proliferation of embryonic rodent spinal cord stem cells compared to FGF-2 alone.

All publications mentioned in the above specification are herein incorporated by reference. Various modifications and variations of the described methods and system of the present invention may be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. An isolated or purified mitogen obtainable from a human tumour cell line.

2. Conditioned medium comprising a mitogen, said medium being conditioned via the growth of a human tumour cell line therein.

3. Conditioned medium comprising a mitogen, said medium being conditioned via the growth of a human tumour cell line.

4. An isolated or purified mitogen obtained from a human tumour cell line.

5. A mitogen obtainable from the medium in which a human tumour cell line has been cultured.

6. A stem cell mitogen obtainable from human tumour cell line.

7. A stem cell mitogen obtainable from the medium in which a human tumour cell line has been cultured.

8. A neural stem cell mitogen obtainable from human tumour cell line.

9. A neural stem cell mitogen obtainable from the medium in which a human tumour cell line has been cultured.

10. A mitogen according to any one of the preceding claims wherein the mitogen is NSC-MF-1 and/or NSC-MF-2.

11. A mitogen according to any one of the preceding claims wherein the mitogen is obtainable from a method comprising the use of sequential Sepharose 4B and anion exchange chromatographic techniques to at least partially purify the mitogen from other contaminating proteins.

12. A method for culturing eukaryotic cells in vitro in a medium comprising a mitogen according to any previous claim.

13. A method for culturing eukaryotic cells in vitro in a medium comprising an exogenously added mitogen according to any previous claim.

14. A method for culturing eukaryotic cells in vitro in a medium comprising exogenously added conditioned medium according to any previous claim.

15. A method for promoting cell survival comprising culturing said cell in medium comprising an exogenously added mitogen according to any previous claim.

16. A method for promoting cell survival comprising culturing said cell in medium comprising exogenously added conditioned medium according to any previous claim.

17. A mitogen according to any previous claim wherein said mitogen is a protein or fragment thereof.

18. A mitogenic composition comprising a mitogen according to any previous claim, and/or comprising conditioned medium according to any previous claim.

19. A method for the preparation of a mitogenic composition comprising
(i) providing a suitable diluent
(ii) adding a mitogen and/or conditioned medium according to any previous claim thereto.

20. A method for modulating the mitogenic effect of a first mitogen comprising adding a suitable amount of a second mitogen according to any previous claim.

21. A method for augmenting the mitogenic effect of a factor selected from GMCSF, LIF, EGF, FGF, IGF-1, FGF-8, thrombopoietin or neurotrophin-3, comprising adding an augmenting amount of a mitogen according to any previous claim.

22. A method for propagating a cell, said method comprising incubating said cell in a medium comprising a mitogen according to any previous claim.

23. A method for potentiating neural cell growth as shown in the accompanying drawings comprising the use of a mitogen according to the invention as described herein.

24. Use of a mitogen according to any previous claim to selectively potentiate the proliferation of progenitor cells that give rise to an increase in neurons.

25. A method of increasing the proportion of neurons developing from a population of neural stem cells, said method comprising contacting said population of neural stem cells with a mitogen according to any previous claim.
